(19) United States
(12) Patent Application Publication
(10) Pub. No.: US 2009/0197819 A1
(43) Pub. Date: Aug. 6, 2009

(54) Compositions for improving and repairing skin

(76) Inventors: Clarence Albert Johnson, Wyoming, MN (US); John A. St. Cyr, Coon Rapids, MN (US)

Correspondence Address: Kathleen R. Terry, 13840 Johnson Street NE, Ham Lake, MN, 55304, US

(21) Appl. No.: 11/726,020

(22) Filed: Mar. 20, 2007

(51) Int. Cl. A61K 31/7004

(52) U.S. Cl. 514/23

(57) ABSTRACT

Compositions for improving the condition of the skin or for repairing damaged skin are disclosed. The compositions contain D-ribose as the active ingredient plus a penetrant to deliver D-ribose to the basal cells and dermis of the skin. Compositions further containing emollients and emulsifiers are disclosed.

### BACKGROUND OF THE INVENTION

The skin is the interface between an organism and the external environment. As such, it is continually subject to stresses such as extreme heat or cold, attack by microorganisms, exposure to UV radiation, abrasion, chemical irritants and the like. As a result, the skin shows signs of damage such as sunburn, roughening, wrinkling, loss of tone, discoloration, and even malignancies, including basal cell carcinoma, squamous cell carcinoma and melanoma. While these effects are often considered to be normal aging, in fact, they are not normal results of aging but are responses to damage.

Many of the effects may be related to free radical formation or suboptimal cell function. The skin is an organ with a high metabolic rate and a high cell turnover. Because it serves as a barrier to microbes, chemicals, radiation, heat and water, skin is highly impermeable. The dermis or corium layer contains cells, blood vessels and nerves. It projects into the epidermis or outer layer in ridges called papillae. The cells of the dermis secrete a ground substance and collagen that give support to the epidermis. The dermis also contains hair follicles and oil glands.

The epidermis is composed of five layers. The basal layer cells are columnar and arranged perpendicularly. These cells divide rapidly to provide a continually renewing epidermis. The basal layer is also the site of melanin formation. As the layers are pushed up towards the surface of the skin by the formation of new cells, they become progressively more dehydrated and keratinized to form the stratum spinosum, flattened polyhedral cells with short processes or spines; the stratum granulosa, composed of flattened granular cells; the stratum lucidum, composed of several layers of clear transparent cells in which the nuclei are indistinct or absent, and the stratum corneum or cornified layer. This layer is comprised of flattened, dead, keratinized cells that form a barrier to the external environment. As the final step in the ever-renewing metabolism of the skin, the stratum corneum gradually flakes off. Integrity of the skin requires good function in all layers.

In damaged skin, the dermis may secrete less collagen or the collagen may become cross-linked by free radicals or radiation and lose elasticity, resulting in sagging and wrinkling. UV radiation causes the stimulation of the basal cells to produce protective melanin. While a tanned skin is considered a sign of health, actually, a tan is a response to UV damage.

During the teen years and even the adult years, the hair follicles or oil glands may become overstimulated, becoming plugged and infected, resulting in the condition termed acne in teens and adult-onset acne or Rosaceae in adults. The site of acne is generally the face, although in severe cases, the condition can afflict the back. Control of acne generally involves topical or systemic administration of astringents such as retinol-type compounds which have the effect of drying up oil-producing glands. The infection may be controlled by topical application of lotions or creams containing astringents and antibacterial agents.

Finally, some skin may have an epidermis that is unnaturally dry and flaky, possibly because of sub-optimal turnover of the dermal cells. Besides detriments to health in having a less functional barrier to infection, damaged skin is less aesthetically pleasing. Unfortunately, the exact areas that are most exposed to the environment and sustain the most damage are those that the subject presents to the world, that is, the face and hands.

Many products have been marketed to improve skin function and appearance. A popular preparation is a cream that incorporates alpha-hydroxy acids, which appear to function as mild irritants that stimulate the exfoliation of the most external stratum corneum, thereby exposing the less cornified and less dry-appearing layers. Unfortunately, many subjects find that these acids are too irritating and may actually cause a red, rough rash. Anti-inflammatory corticosteroids such as hydrocortisone are often used to soothe irritated skin and eliminate unwanted redness. U.S. Pat. No. 6,242,491 discloses that mammals fed creatine show decreased free radical damage in striatal tissue, which is extrapolated to improve skin condition.

Some efforts have been made to improve the condition of skin by applying nutrients topically, on the theory that these nutrients will penetrate the dermis and speed up the turnover of dermal cells, thus presenting younger, more youthful appearing cells to the surface. An example is U.S. Pat. No. 5,053,230, issued Oct. 1, 1991, in which a nutrient medium previously found to support in vitro culture of human epithelial cells was extrapolated to in vivo use to promote the trophism of the skin and related follicles. This complex mixture contains amino acids, monosaccharides, nucleosides and vitamins. Simpler compositions containing sucrose are on the market.

The need remains for a composition to be applied topically to stimulate the growth of epidermal and dermal cells, thereby improving the condition of the skin and repairing damaged skin.

### SUMMARY OF THE INVENTION

D-ribose, or ribose-related compounds, are administered to a mammal as a topical preparation. Ribose-related compounds include D-ribulose, 5-phospho-ribose, xylulose, xylitol, inosine and sedoheptulose.

The effective agent at a concentration of 0.1 to 5% combined with a penetrant and incorporated into a pad, a lotion or a cream. The effective agent is most preferably combined with a penetrant and an emollient in a pharmaceutically acceptable carrier. A bacteriostatic agent can be added. The composition is sprayed or smoothed onto the areas of the skin to be treated, either directly or applied with a pad. The composition is preferably applied daily, most preferably twice or thrice daily, to skin that is to be treated.

If acne, rosaceae or other skin infection is to be treated topically, an astringent and/or a antibacterial agent is added. The preferred astringent is retinol or a peroxide. The most preferred peroxide is benzoyl peroxide. Peroxide may act as a antibacterial agent as well as an astringent, or an antibiotic such as neosporin or bacitracin may be added.

A preservative may be added to a lotion or cream. Common preservatives include formalin, methylchloroisothiazine, methylisothiazolinone, butylated hydroxy toluene, and EDTA.

### DETAILED DESCRIPTION OF THE INVENTION

It is known that ATP plays an essential role in the function of all cells. The level of ATP produced may be too low in respect to the requirements of the cells. ATP molecules are constantly recycled in order to furnish the necessary energy. When skin cells are exposed to different stresses, including pollution, smoking, physical exertion, aging, etc., the cells require more energy than can be recycled. The level of ATP may be decreased, affecting cellular metabolism. It has previously been shown that the oral administration of D-ribose raises ATP levels in skeletal muscle (U.S. Pat. Nos. 6,159,942 and 6,534,480). Equally important as its role in ATP formation, D-ribose is its role in the synthesis of DNA and RNA for cell division and growth and for the formation of cyclic AMP and other cell signals.

Ribose-related compounds are those compounds which are structurally related to D-ribose, or which are readily converted to D-ribose in vivo. The group includes D-ribulose, 5-phospho-ribose, xylose, xylulose, xylitol, inosine and sedoheptulose. All of these have been previously shown to have similar, although reduced effects as D-ribose.

The effective agent, D-ribose being the exemplar selected, is used at a concentration of 0.01 to 10%, preferably from 0.1 to 5% by weight D-ribose, and most preferably from 0.1 to 1.5% by weight D-ribose, in respect to the total weight of the composition. The D-Ribose can be administered as a spray in water, but is more preferably combined with a penetrant and incorporated into a lotion or cream. The effective agent is most preferably combined with a penetrant and an emollient in a pharmaceutically acceptable carrier. A bacteriostatic agent can be added. The composition is smoothed onto the areas of the skin to be treated, either directly or applied to a cloth pad. The composition is preferably applied daily to skin that is exposed to the external environment, but more preferably two or three times a day.

The topical compositions of the present invention are formulated as solutions or emulsions in a pharmaceutically acceptable carrier. Such a carrier is based on a solvent which is capable of dissolving the active ingredient and which is known to be non-toxic and non-irritating. Such solvents include water, dilute solutions of 1-10% alcohol in water, propylene glycol, glycerol, and mixtures thereof. Acceptable alcohols include ethanol, propanol and butanol. The composition will contain from about 0.5 to 50% w/v active ingredient.

Lotions and creams are frequently applied to maintain the surface texture of skin. It would be simple and efficacious to include the active ingredient in any commercially available lotion or cream. This invention provides typical formulations of such a lotion or cream.

Since the object of the invention is to deliver active ingredient to the basal and dermal cells below the epidermis, when the skin barrier is intact, it is necessary to include a penetrant in the solutions, lotions or creams of this invention. Such a penetrant will enhance the delivery of active ingredient by transitory disruption of the cell to cell connections of the skin. Penetrants in general include ionic detergents, non-ionic detergents, fatty acids, organic solvents and oils. Certain constructs such as liposomes and nanoparticles can also be used to transport active ingredient through the stratum corneum barrier. It is well known in the industry to select a penetrant with the desired characteristics, which vary in effect from the intense penetration of the solvent dimethyl sulfoxide (DMSO) which cause the rapid co-transport past the skin barrier of any agent present on the skin, to the milder action of dilute ethanol. Of recent interest are the constructs termed liposomes and nanoparticles, which are molecules that form closed structures that can incorporate an active ingredient. Liposomes and nanoparticles can readily penetrate otherwise impermeable barriers.

Acne is a skin condition that is characterized by purulent infection of the hair follicles or oil glands. Acne is treated with topical or systemic antibiotics or with retinoids, which inhibit oil production in oil glands. Milder cases may be controlled by over the counter medications containing peroxides such as benzoperoxide. The active ingredients of this invention are beneficial adjuvants in the control of acne for two effects. First, the general improvement in the condition of the skin, with increased energy and better circulation, is beneficial to health. As disclosed in U.S. Pat. No. 6,663,859, the teachings of which are incorporated by reference, D-ribose stimulates the immune response, which can augment antibacterial compounds in order to eliminate the infection seen in acne or rosaceae. The stimulation continues past the infective stage to enable the skin to heal, preferably without scarring or pitting.

### DEFINITIONS

“Ribose” for the purposes of this invention, means D-ribose and ribose-related compounds D-ribulose, 5-phospho-ribose, xylose, xylulose, xylitol, inosine and sedoheptulose.

“Penetrants” include any compound or construct that enhances the passage of the effective agent through the epidermis to the basal and dermis layers of the skin. Penetrants include but are not limited to, ethanol, butanol, propanol, myristyl alcohol, lauryl sulfate, DMSO, liposomes and nanoparticles.

“Emollients” include materials used for the prevention or relief of dryness and flaking of the epidermis. Emollients include lanolin, mineral oil, glycerin, high molecular weight alcohols such as cetyl alcohol, vegetal oils, animal fats, and the like. Emollients may also have a penetrant effect

“Pharmaceutically acceptable carrier” means any combination of solvent and additives that effectively suspend the active ingredient. The invention is not limited to any composition or compositions. If the effective ingredient alone is applied, water plus a penetrant is a pharmaceutically acceptable carrier. If the composition includes emollients, an emulsifier is required to hold the emollient in suspension. Emulsifiers include surface active agents such as polysorbate 20 sodium stearyl lactate, glyceryl dilaurate, sorbitol, isopropyl myristate and sodium lauryl sulfate.

“Preservative” includes any bactericidal or bacteriostatic agent commonly used to present bacterial growth in compositions for topical use. Common preservatives include formalin, methylchloroisothiazine, methylisothiazolinone, butylated hydroxy toluene, EDTA and the like.

“Stabilizers” are included. Chelators stabilize the emulsion. Chelators include EDTA and citrate. Other stabilizers include aluminum stearate, sorbitol, dimethicone, and polyethylene glycol.

“Thickeners” are added to control the consistency of the lotion or creme. The texture of the product, from flowable lotion to semi-solid cream, is readily varied by adding thickeners, which are generally higher molecular weight animal fats and animal fat compounds such as oleates, stearates, glycol stearate, and stearyl acetate. Waxes such as cetyl alcohol can be used.

Collagen and hyaluronic acid may be added.

The following examples are provided to further explain how to make and use the invention, but are not limitations on the scope of the invention.

### Example 1

### Effect of Ribose on Cultured Fibroblasts

Applicants have previously shown (U.S. Pat. No. 6,663,859) that ribose stimulates the growth of lymphocytes in cell culture. Based on this information, Applicants believe that the same effect might be shown in cell culture with skin cells. The first skin cell be tested in vitro was the fibroblast, the culture of which is well known. The Coriell Institute for medical research publishes a web site with detailed instructions, the teachings of which are incorporated by reference. (Http:/locus.umdnj.edu/cer/faq/fibro.html.) Fibroblast cell cultures were tested to determine an optimal level of ribose as compared to glucose control.

To determine a protective effect of ribose, test and control plates were grown to about 80% confluence and subjected to a simulated environmental stress, six hours under an hypoxic atmosphere. Following exposure, the medium was aspirated off the culture and fresh medium applied. Cultures were inspected at 12, 24 and 48 hours to determine recovery of ATP to pre-hypoxia levels. Results of the first experiment showed a partial recovery to 19% of base ATP levels in the ribose-treated cells versus no recovery in the glucose control cells.

### Example 2

### Simple Solution of Ribose

Ribose (most frequently chosen to be D-ribose as an exemplar pentose compound) at a concentration of 5% will be dissolved in water plus 1-10% ethanol. The composition will be provided as a solution, preferably in a spray bottle. In use, a spray will be applied on the areas to be treated, most usually the face and hands or a damaged area. Alternatively, the effective ingredient will be incorporated in a dry form in a pad. In use, the subject will moisten the pad with water or a solution of 1-10% ethanol in water and apply the pad to the area to be treated. The pad may be secured with adhesive tape and will preferably be applied for several hours, preferably overnight, in order to provide sustained treatment.

### Example 3

### Composition with Penetrant

The skin is a barrier. Preliminary effects to determine the effect on improving skin by the application of ribose were unsuccessful, leading the Applicants to the realization that ribose may not have penetrated to the basal cells and dermis. To get penetration it is advantageous to provide a penetrant to increase the concentration of active ingredient at the dermis and epidermis. A preferred penetrant is ethanol, which will be incorporated into the composition at a concentration of 0.1 to 15%, most preferably 10% weight to volume. The active ingredient may be at a concentration of from 0.5 to 10%, preferably 1%, weight to volume.

### Example 4

### Preliminary Trial to Test Dermal Cell Turnover in Normal Skin

A cohort of 40 women between the ages of 16 and 30 will be entered into a clinical study. Exclusion criteria are:

Active skin diseases including eczema, contact dermatitis, psoriasis or severe acne.

Pregnancy or lactation.

Administration of steroids or antibiotics within previous six months.

Each subject will serve as her own control. A pulse of 10 ml of 1% fluorescein in 10% ethanol will be applied to the inner lower arm, four inches below the elbow. The fluorescein will be swabbed on each arm with a cotton swab and gently massaged into an area about two inches in diameter. After the fluorescein is allowed to penetrate the skin for 20 minutes, the area is swabbed with a mild detergent solution until the wash swab does not fluoresce when black light (380 nm) is applied. The subjects are divided into two groups. One group is swabbed on the right arm with 5% ribose in 10% ethanol, covering the area of application of fluorescein and extending one inch beyond that area. The left arm is swabbed with 5% glucose in 10% ethanol and serves as a control. The other group is treated in a similar manner with 5% ribose in 10% ethanol plus emollients or 5% glucose in 10% ethanol plus emollients. The respective compositions are applied four times a day for one month on the test area only. The subjects are asked to refrain from using any perfumed soaps, any cosmetics or lotions on their arms for the test period and are advised to avoid sunlight on the treated areas. An initial reading is taken and the fluorescence on the surface of the skin is followed. The readings are rated as follows:

1. Bright fluorescence.
2. Fluorescence, uniform but dim.
3. Fluorescence, not uniform, dim.
4. Fluorescence only in isolated spots.
5. No fluorescence.

It is known that the fluorescein will penetrate to the lower layers of the dermis and will bind covalently to the cell membrane proteins in all the layers. Therefore, with time, as the epidermis is replaced and sloughed off, the fluorescence will diminish, giving an estimate of cell turnover. It is expected that the areas of skin that have been treated with the test compositions containing ribose will lose fluorescence more rapidly than the control areas, indicating that ribose plus a penetrant has stimulated the turnover of dermal cells.

### Example 5

### Lotions and Creams with Ribose

In actual practice, a composition is more convenient and acceptable for use when the ribose is incorporated in a composition than includes other conditioning agents, such as emollients and oils. Lotions and creams for topical application differ only in consistency, the ingredients being essentially the same. A cream may be made according to the composition for a lotion by decreasing the water content, by adding a thickening agent or by replacing lower molecular weight fats with higher molecular weight fats or waxes. Color or a whitening agent, fragrance, vitamins, and the like are often added. U.S. Pat. Nos. 4,939,179 and 4,309,448, the teaching of which are hereby incorporated by reference, disclose compositions that have been accepted in the market place. Three compositions for a lotion and a cream according to this invention are:

COMPOSITION A

| Ingredient | Concentration, % | Primary Function |
|---|---|---|
| D-ribose | 1 | Stimulate cell turnover |
| Mineral Oil | 10 | Emollient |
| Isopropyl myristate | 3 | Emulsifier |
| Polyethylene glycol-40 | 4 | Thickener |
| Sorbotan peroleate | 1 | Emollient, Stabilizer |
| Propylene glycol | 1 | Penetrant |
| Cetyl palmitate | 10 | Emollient, Thickener |
| Lanolin alcohol | 10 | Emollient, Thickener |
| Butylated | 0.2 | Preservative |
| hydroxytoluene | | |
| Aluminum Stearate | 4 | Emollient, Stabilizer |
| Sodium lauryl sulfate | 2 | Emulsifier |

COMPOSITION B

| Ingredient | Concentration, % | Primary Function |
|---|---|---|
| D-ribose | 1 | Stimulate cell turnover |
| Glycerin | 8 | Emollient |
| Cetyl esters | 2 | Emollient, Thickener |
| Lanolin oil | 6 | Emollient |
| Sorbitol | 1 | Emulsifier, Stabilizer |
| Dimethicone | 7.5 | Stabilizer |
| Methylparaben | 4 | Thickener |
| Propylparaben | 4 | Thickener |
| SD-alcohol-40-B | 1 | Penetrant |
| Glyceryl dilaurate | 2 | Emulsifier |
| Butylated | 0.2 | Preservative |
| hydroxytoluene | | |

COMPOSITION C

| Ingredient | Concentration, % | Primary Function |
|---|---|---|
| D-ribose | 1 | Stimulate cell turnover |
| Glycerin | 4 | Emollient |
| Stearic acid | 4 | Emollient, Thickener |
| Glycol stearate | 2 | Emollient, Penetrant |
| Lecithin | 2 | Emulsifier |
| Corn oil | 10 | Emollient |
| Collagen | 1 | Stabilizer, Nutrient |
| Disoldium EDTA | 0.5 | Emulsifier |
| Titanium dioxide | 0.1 | Colorant |
| Yellow dye 10 | 0.05 | Colorant |
| Sodium stearyl lactate | 3 | Emollinet, Emulsifier, |
| | | Penetrant |

In each of these compositions, the ingredients are dissolved or suspended in water. D-ribose may be replaced by ribose-5-phosphate, xylose, ribulose, xylitol, inosine or sedoheptulose.

### Example 6

### Use of Liposomes as a Penetrant

Liposomes are micellar structures comprised of amphoteric molecules that form an enclosed space. Many cosmetic preparations, especially so-called antiwrinkle creams, encapsulate oils within liposomal micelles. It has been found that the lipids readily form an attachment with cell membranes.

### Example 7

### Compositions for Treating Acne

The active ingredients are combined with an astringent and/or peroxidizing agent such as benzoyl peroxide. Most preferably, these compositions are included in a “towelette,” that is, a small moist pad. The towelette is rubbed gently over the affected areas several times a day.

Those skilled in the art can readily make substitutions and additions to the exemplar compositions disclosed within without departing from the spirit and scope of the appended claims.

We claim:

1. A composition comprising an effective amount of D-ribose and a penetrant dissolved in water.

2. The composition of claim 1 wherein the effective amount of D-ribose is 0.1% to 10% weight to volume.

3. The composition of claim 1 wherein the effective amount of D-ribose is 0.5% to 1.5% weight to volume.

4. The composition of claim 1 wherein the penetrant is an alcohol, a surface active agent or an emollient.

5. A composition comprising an effective amount of D-ribose, a penetrant, an emollient and an emulsifier.

6. The composition of claim 5 wherein the effective amount of D-ribose is 0.5 to 1.5%, the penetrant if an alcohol, a surface active agent or an emollient.

7. The composition of claim 6 wherein the emollient is a mineral oil, a vegetal oil, an animal fat or an alcohol derivative of a vegetal oil or an animal fat.

8. The composition of claim 7 wherein the animal fat is an oleate or a stearate.

9. The composition of claim 6 wherein the emulsifier is polysorbate 20, sodium lauryl sulfate, sodium stearyl lactate, glyceryl dilaurate, sorbitol and isopropyl myristate.

10. A method to improve the condition of the skin comprising the topical application of the compositions of claim 1 daily for at least a week.

10. A method to improve the condition of the skin comprising the topical application of the compositions of claim 5 daily for at least a week.
